(19) United States
(12) Patent Application Publication
(10) Pub. No.: US 2002/0150598 A1
(43) Pub. Date: Oct. 17, 2002

(54) COLLAGEN INCORPORATING COSMETICS

(76) Inventors: YUJIRO HAMADA, TOKYO (JP); KOKICHI SHIRAI, TOKYO (JP); CHIKA SATO, TOKYO (JP); SHINKICHI IRIE, TOKYO (JP); SHUNJI HATTORI, TOKYO (JP); MASARU NISHIZAWA, TOKYO (JP); MASAO TAZAKI, TOKYO (JP)

Correspondence Address: BROWDY AND NEIMARK, P.L.L.C., 624 NINTH STREET, NW, SUITE 300, WASHINGTON, DC, 20001-5303, US

(21) Appl. No.: 09/427,672

(22) Filed: Oct. 27, 1999

(30) Foreign Application Priority Data
Oct. 28, 1998 (JP) 306786/1998

(51) Int. Cl. A61K 7/00

(52) U.S. Cl. 424/401

(57) ABSTRACT

Cosmetics incorporating a cosmetic feed containing a dispersion of a collagen association product that is obtained by adjusting an aqueous solution of collagen having a specified isoelectric point to a pH near the isoelectric point.

### BACKGROUND OF THE INVENTION

This invention relates to a cosmetic feed and cosmetics incorporating the feed. More particularly, the invention relates to a cosmetic feed containing a dispersion of a collagen association product having high thermal denaturation temperature and collagen containing cosmetics of high thermal denaturation temperature that incorporate the feed.

Cosmetics containing water-soluble collagen are currently on the market as useful skin care products having a moisture retaining function. Water-soluble collagen is usually produced from animal skin, hides and bones either by extracting the small contents of soluble collagen with dilute acids (this type of collagen is commonly called “acid soluble collagen” and hereinafter sometimes abbreviated as “ASC”) or by solubilizing them with an enzyme (this type of collagen is commonly called “solubilized collagen with enzyme” and hereinafter sometimes abbreviated as “SCE”) or an alkali (this type of collagen is commonly called “solubilized collagen with alkali” and hereinafter sometimes abbreviated as “SCL”).

Of the three types of collagen mentioned above, ASC and SCE become turbid in the pH range of 5.5-7.5 which is typical for cosmetic formulations on account of collagen's refibrogenesis or isoelectric precipitation. In order to avoid this problem and enhance collagen's solubility, transparency or miscibility with other components, chemical modification is currently practiced to shift the isoelectric point of collagen molecules so that it is more acidic than pH 5.5 or more alkaline than pH 7.5 (for chemical modification by esterification, see Unexamined Published Japanese Patent Application (kokai) No. 27192/1996, and for chemical modification by succination, see Unexamined Published Japanese Patent Application (kokai) No. 28947/1980).

The three types of water-soluble collagen have a common problem in that they have low thermal denaturation temperature in solution, particularly when the solution is transparent with high water content. Stated specifically, the collagen chemically modified after production by acid extraction or enzyme processing starts to undergo thermal denaturation at 35-37° C. which peaks at 37-40° C. within the pH range of cosmetics into which an aqueous solution of the collagen is incorporated, typically between 5.5 and 7.5; the corresponding temperatures for the water-soluble collagen produced by alkali processing are about 30° C. and 34-35° C., respectively (see FIG. 1 and Table 1). If cosmetics incorporating aqueous solutions of these types of collagen are heated to temperatures higher than their thermal denaturation start points, the triple helix structure which is essential for them to function as collagen degrades gradually to undergo irreversible thermal denaturation until their physical and biochemical properties change so significantly that the cosmetics can no longer maintain the desired quality.

Therefore, cosmetics that use the water-soluble collagen in a solubilized form require thorough control of low temperatures at all stages including manufacture, storage and distribution and this is a substantial factor to increase the overall cost of cosmetics production. Particularly great care is needed when using such cosmetics in summer.

### SUMMARY OF THE INVENTION

The present invention has been accomplished under these circumstances and has as an object overcoming the aforementioned defects of the conventional water-soluble collagen and providing a collagen-based cosmetic feed that can be incorporated into cosmetics with the thermal denaturation temperature of collagen held at high level.

Another object of the present invention is to provide cosmetics incorporating collagen with its thermal denaturation temperature held at high level.

In the course of their intensive efforts to increase the thermal denaturation temperature of collagen, the present inventors prepared collagen in such a way that its isoelectric point was equal to the pH commonly used in cosmetic formulations (e.g. pH 5.5-7.5) and found that an association product obtained by adjusting an aqueous solution of the thus prepared collagen to a pH near its isoelectric point had a significantly high thermal denaturation temperature. The inventors also found that the association product had the right characteristics that enabled it to be incorporated into cosmetics. The present invention has been accomplished on the basis of these findings.

In one aspect, the present invention provides a cosmetic feed containing a dispersion of a collagen association product that is obtained by adjusting an aqueous solution of collagen having a specified isoelectric point to a pH near the isoelectric point.

In another aspect, the present invention provides cosmetics incorporating a cosmetic feed containing a dispersion of a collagen association product that is obtained by adjusting an aqueous solution of collagen having a specified isoelectric point to a pH near the isoelectric point.

### BRIEF DESCRIPTION OF THE DRAWING

FIG. 1 is a diagram showing the thermal denaturation temperatures of three kinds of collagen as measured by differential scanning calorimetry (DSC).

### DETAILED DESCRIPTION OF THE INVENTION

If the cosmetic to be prepared has a final pH of 7.0, collagen is chemically modified to have an isoelectric point of 7.0 and its pH is adjusted to a value near 7.0 to yield an association product, which is dispersed for incorporation into cosmetics. The term “collagen association product” as used herein means the product of so-called “isoelectric precipitation” from an aqueous solution of collagen that has its pH adjusted to a value near its isoelectric point, whereupon the apparent electric charges on the collagen molecules become zero, causing the collagen molecules to associate with themselves to form a white precipitate.

While collagen has heretofore been used as a component in cosmetics of high water content, a demand has arisen to increase its transparency. To meet this need, various chemical modifications are performed on collagen so that it remains water-soluble in the typical pH range for cosmetic formulations. However, the collagen molecules in solution are heat labile and cosmetics incorporating them are by no means heat stable. Water-insoluble association products of collagen having isoelectric points in the pH range for cosmetic formulations are also held unsuitable for use as cosmetic feeds for several reasons such as the milk white color and inhomogeneity they present with. To deal with this difficulty, the aforementioned efforts have been made to prevent the formation of the association products of collagen. However, it has been found that the collagen association product of the invention exhibits cell adhesive activity and other characteristics that are by no means inferior to those of the conventional cosmetic-grade collagen. In addition, if dispersed mechanically as with a homogenizer, the collagen association product becomes sufficiently suitable for use as a cosmetic feed. What is more, cosmetics incorporating the dispersion of the collagen association product have sufficient heat stability to justify their commercial use. The dispersion of the collagen association product starts to undergo thermal denaturation at 42-43° C., which peaks at 48-49° C.; thus, it shows a significantly high heat stability compared with the conventional collagen.

In the present invention, an association product of collagen may be prepared from any types of insoluble collagen fibers that may originate from all sources including mammals such as bovine and swine, as well as birds and fishes.

Known methods for the production of water-soluble collagen include (1) extraction with dilute acids, (2) use of a protease (see, for, example, Examined Japanese Patent Publications (kohkoku) Nos. 1175/1969 and 11037/1969) and (3) use of aqueous alkali solutions (see, for example, Examined Japanese Patent Publication (kohkoku) No. 15033/1971).

These methods are specifically described below. In an exemplary process for the preparation of insoluble collagen fibers, lime-processed splits are washed, shredded to squares of about 10 cm with a ham slicer, minced and further ground down mechanically, followed by degreasing with an organic solvent (e.g. acetone-ether) or a lipase, and thorough washing.

(1) Extraction with Dilute Acid

The insoluble collagen fibers prepared by the method described above are suspended in 10 volumes of 0.5 M acetic acid and extracted at 4° C. for 72 h with stirring or shaking. The residue is removed by centrifugation. After purification by repeated cycles of salting-out (a salt such as sodium chloride is added to give a final concentration of 5%) and dissolution (the salted-out precipitate is re-dissolved in the aqueous solution rendered acidic with acetic acid or the like), desalting is performed by, for example, dialysis or isoelectric precipitation to yield acid soluble collagen (ASC).

(2) Solubilization with Enzyme

The insoluble collagen fibers prepared by the method described above are suspended in distilled water to give a final collagen concentration of 2% and adjusted with HCl to pH 3. An acidic protease is added in an amount of a hundredth of the collagen weight and solubilization is performed at 25° C. for 72 h. After quenching the enzyme reaction, purification is performed by repeated cycles of salting-out (a salt such as sodium chloride is added to give a final concentration of 5%) and dissolution (the salted-out precipitate is re-dissolved in the aqueous solution rendered acidic with HCl or the like) and by filtering with a cloth, filter paper, a metal mesh screen or the like. Then, desalting is performed by, for example, dialysis or isoelectric precipitation to yield solubilized collagen with enzyme (SCE).

(3) Solubilization with Alkali

Sodium hydroxide and monomethylamine are mixed in water to prepare a solubilizing aqueous solution having respective final concentration of 3% and 1.9% (V/W). Insoluble collagen fibers conditioned to have a final collagen concentration of 4.5% are suspended in the solubilizing aqueous solution and solubilization is performed at 18° C. for 3 weeks. After quenching the solubilization reaction, purification is performed by repeated cycles of salting-out (a salt such as sodium chloride is added to give a final concentration of 5%) and dissolution (the salted-out precipitate is re-dissolved in the aqueous solution rendered acidic with HCl or the like) and by filtering with a cloth, filter paper, a metal mesh screen or the like. Then, desalting is performed by, for example, dialysis or isoelectric precipitation to yield solubilized collagen with alkali (SCL).

The collagen association product having high thermal denaturation temperature according to one aspect of the invention is obtained by first preparing water-soluble collagen having an isoelectric point near the final pH of a cosmetic into which the association product is to be incorporated and then adjusting the water-soluble collagen to have said pH. The inherent isoelectric point of ASC and SCE is at pH 8-9 and that of SCL is at pH 4.5-5.0. If the association product is to be incorporated into a cosmetic having a final pH of 7.0, ASC and SCE are chemically modified such that their isoelectric point is shifted toward the acidic side whereas SCL is chemically modified such that its isoelectric point is shifted toward the alkali side. Thus, either type of collagen is adjusted to have an isoelectric point near pH 7.0.

In order to shift the isoelectric point toward the acidic side, known methods of chemical modification for increasing the negative electric charges on collagen molecules may be used, as exemplified by succination (see Unexamined Published Japanese Patent Application (kokai) No. 28947/1980). Another method that can be used is the above-described solubilization with alkali.

In order to shift the isoelectric point toward the alkali side, known methods of chemical modification for increasing the positive electric charges on collagen molecules may be used, as exemplified by esterification (see Unexamined Published Japanese Patent Application (kokai) No. 27192/1996) and amidation. The skilled artisan may choose an appropriate method of chemical modification in accordance with the type of the water-soluble collagen feed used and perform routine simple tests to prepare collagen having the desired isoelectric point.

When the collagen thus adjusted to have the desired isoelectric point is incorporated in a cosmetic, the collagen molecules undergo isoelectric precipitation to form an association product, which eventually increases the thermal denaturation temperature of the cosmetic.

Collagen may be incorporated into cosmetics by either one of the following methods: a collagen solution having the desired isoelectric point is added to an aqueous solution adjusted to have a pH equal to the isoelectric point of collagen, whereupon the collagen molecules undergo isoelectric precipitation to form an association product, which is dispersed uniformly in a medium and incorporated into a cosmetic of interest; alternatively, the collagen solution having the desired isoelectric point is incorporated as such into a cosmetic, whereupon the collagen molecules undergo isoelectric precipitation to form an association product which is subsequently dispersed uniformly. Any method of dispersing known in the art may be employed and a preferred method is a mechanical one using a homogenizer or the like.

The collagen association product according to one aspect of the invention may be incorporated in cosmetics in any amounts as long as cosmetic preparations are obtained. To give guide figures, the association product as calculated for collagen's dry weight may be incorporated in amounts of 0.01-2%, preferably 0.1-1.5%, more preferably 0.5-1.0%, of cosmetic's weight, although these ranges are variable with the type of cosmetics.

Besides the collagen association product, the cosmetic according to the second aspect of the invention may contain other additives as appropriate for its specific form; examples are preservatives such as paraoxybenzoate esters (parabens), metal sequestrants such as a sodium salt of ethylenediaminetetraacetic acid (EDTA) and polymeric thickening agents such as gums which are polysaccharide based natural polymers.

The cosmetic according to the second aspect of the invention may take all forms that are available in the conventional collagen-incorporating cosmetics, as exemplified by lotions, creams, emulsion, jellies and essences.

### EXAMPLES

The following examples are provided for the purpose of further illustrating the present invention. It should, however, be noted that the conditions set forth in the examples are in no way to be taken as limiting the invention and that the skilled artisan will understand that various improvements and modification of the methods described below may be made without departing from the spirit and scope of the invention.

In the examples that follow, all percentages that represent the amounts of various ingredients are on a weight basis unless otherwise indicated.

### Example 1

Insoluble Collagen Fibers as a Feed

The corium layer of an adult cattle hide was ground with a mincer or the like, degreased and washed thoroughly to prepare a feed.

Solubilizing Insoluble Collagen Fibers with Enzyme

The collagen feed was dispersed in water to give a collagen concentration of ca. 2.5%. After adjusting the dispersion to a pH of ca. 2.8 with HCl or the like, PROCTASE (a protease produced by Meiji Seika Kaisha, Ltd.) was added in an amount of 1% of collagen's weight and the enzyme reaction was continued with mild stirring at 25° C. for 9 days (the same amount of PROCTASE was added at days 3 and 6). The reaction was quenched by adjusting the dispersion to pH 8-9 with sodium hydroxide.

Preparing Solubilized Collagen with Enzyme Having a Specific Isoelectric Point

Alkali processing was performed. To the prepared solution of solubilized collagen with enzyme, sodium hydroxide and monomethylamine were added to give respective final concentrations of 0.4% and 0.2% (V/W) and the alkali reaction was continued with mild stirring at 18° C. for 3 days. The reaction was quenched by adjusting the solution to a pH of ca. 3 with HCl. In the example under consideration, the isoelectric point of collagen was adjusted to a pH of ca. 7.0 but collagen preparations having isoelectric points at other pH values can be obtained by selecting appropriate reaction temperatures and times.

Purifying Collagen Solution

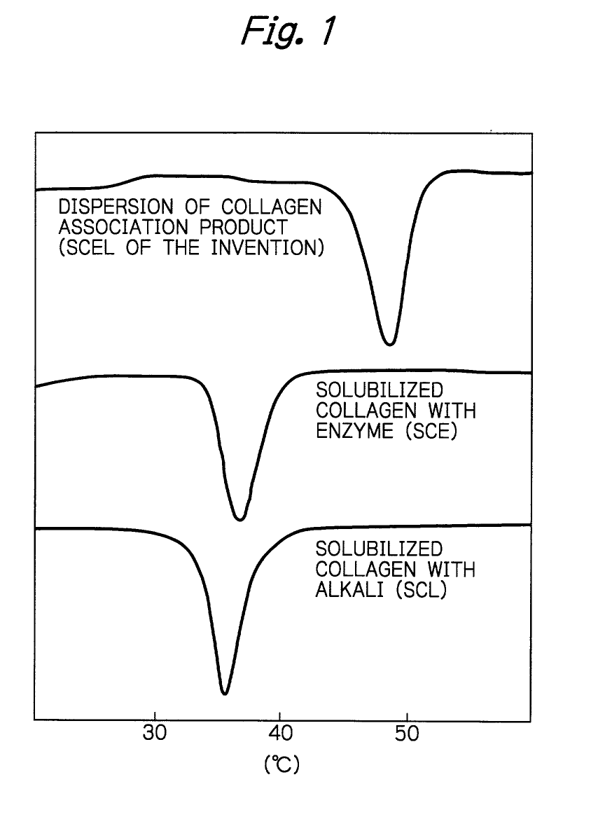

The thus prepared collagen solution having the specified isoelectric point was subjected to salting-out by adding sodium chloride to give a final concentration of 5%. The resulting precipitate was recovered by centrifugation. The recovered precipitate was dispersed in distilled water to give a collagen concentration of ca. 2%. The dispersion was adjusted to pH 3.0 with HCl so that a uniform solution resulted. After filtering with a cloth and filter paper, the solution was adjusted to pH 7.0 with sodium hydroxide so that collagen underwent isoelectric precipitation. The precipitate was recovered by centrifugation, washed again with distilled water adjusted to pH 7.0, and desalted. The purified collagen precipitate was recovered and subjected to the following experiments. The collagen in the recovered precipitate was also analyzed to give the results shown in Tables 1 and 2 and FIG. 1, in which SCEL represents the collagen association product prepared in accordance with the invention. The results for two controls, SCE and SCL, are also shown.

TABLE 1
Analyses of Collagen (SCEL) Forming an Association
Product Having High Thermal Denaturation Temperature

| Item of analysis | SCE | SCL | SCEL |
|---|---|---|---|
| Thermal denaturation | 32.1 | 30.5 | 42.3 |
| start temperature, ° C.*1 | | | |
| Thermal denaturation | 36.5 | 35.4 | 48.7 |
| peak temperature, ° C.*1 | | | |
| Isoelectric point (pH) | 8.8 | 4.9 | 7.0 |
| Percent solubility*2 | ≧95% | ≧95% | ≧95% |
| Optical rotation [α]_{D} *3 | −410˜−420 | −415˜−425 | −410˜−420 |

*1: Temperature reading from the DSC curve in FIG. 1.
*2: Percent passage of 0.3% collagen solution through 0.45 μm membrane filter at pH 3.0.
*3: An index for the integrity of the triple helix structure inherent in collagen. Due to thermal denaturation, the triple helix structure is unfolded to gelatinize the collagen, whereupon the absolute value of optical rotation decreases.

TABLE 2
Amino Acid Composition of Each Collagen
(No. of Amino Acids in 1000 Residues)

| Amino Acid | SCE | SCL | SCEL |
|---|---|---|---|
| Asp | 44 | 44 | 45 |
| Hyp | 97 | 92 | 95 |
| Thr | 16 | 17 | 17 |
| Ser | 35 | 33 | 31 |
| Glu | 76 | 75 | 74 |
| Pro | 131 | 127 | 120 |
| Gly | 333 | 336 | 346 |
| Ala | 110 | 115 | 114 |
| Val | 19 | 20 | 20 |
| Met | 5 | 5 | 2 |
| Ile | 11 | 12 | 12 |
| Leu | 21 | 23 | 23 |
| Tyr | 1 | 1 | 1 |
| Phe | 11 | 11 | 11 |
| Hyl | 6 | 7 | 6 |
| Orn | 0 | 10 | 0 |
| Lys | 28 | 27 | 27 |
| His | 4 | 4 | 4 |
| Arg | 52 | 41 | 52 |
| Total | 1000 | 1000 | 1000 |

The above results show that compared to the conventional types of water-soluble collagen, SCE and SCL, the SCEL of the present invention has a significantly high thermal denaturation temperature at the specified pH commonly used in cosmetic formulations. In addition, SCEL parallels with the conventional types of water-soluble collagen in that it be purified and sterilized by passage through a membrane filter; what is more, it maintains substantially the same amino acid composition as in vivo collagen.

Cell Adhesive Activity of Various Collagen Types

Examination was made to see whether SCEL in the dispersion of the collagen association product having high thermal denaturation temperature exhibited the same degree of cell adhesive activity as the conventional collagen. A collagen sample was coated on a glass plate at a concentration of 10 μg/ml and the degree of human skin keratinocyte adhesion to the collagen sample was examined. The results are shown in Table 3.

TABLE 3
Cell Adhesion to Various Collagen Types

| | Adhesion of Human Skin Keratinocyte | | |
|---|---|---|---|
| Collagen | 30 min | 3 h | 4 days |
| SCEL | +++ | +++ | +++ |
| SCE | +++ | +++ | +++ |
| SCL | ++ | ++ | ++ |
| gelatin | − | + | ++ |
| no coat | − | + | ++ |

(Notes)

SCE: Solubilized collagen with enzyme. Triple helices of collagen molecules were substantially freed of the teropeptide at both terminals by enzyme. In other respects, SCE exhibited substantially the same properties as in vivo collagen.

SCL: Solubilized collagen with alkali. Triple helices of collagen molecules were substantially freed of the teropeptide at both terminals by alkali. In addition, several amino acid species were chemically modified.

gelatin: Gelatin. Irreversibly denatured collagen with heating. Characterized by substantial changes in physicochemical and biochemical properties.

no coat: Non-coated glass plate.

The data in Table 3 shows that even after 4 days of cell culture, SCEL exhibited cell adhesive activity at a substantially comparable level to SCE.

Recognition of Various Collagen Types by Collagen Receptor on Cell

Examination was made to see whether SCEL in the dispersion of the collagen association product having high thermal denaturation temperature would be recognized as collagen by cells, in comparison with the conventional types of water-soluble collagen. A collagen sample was coated on a glass plate at a concentration of 10 μg/ml and human skin keratinocytes were adhered to the coated collagen. The cells were fixed at given time intervals to see how the expression of a collagen receptor (integrin α2β1) progressed. The results are shown in Table 4.

TABLE 4
Recognition of Various Collagen Types
by Collagen Receptor on Cell

| | Collagen Receptor on | | |
|---|---|---|---|
| | Human Skin Keratinocyte | | |
| Collagen | 30 min | 3 h | 4 days |
| SCEL | +++ | +++ | ++ |
| SCE | +++ | +++ | ++ |
| SCL | +++ | + | − |
| gelatin | − | + | − |
| no coat | − | − | − |

(Notes)

SCE: Solubilized collagen with enzyme. Triple helices of collagen molecules were substantially freed of the teropeptide at both terminals by enzyme. In other respects, SCE exhibited substantially the same properties as in vivo collagen.

SCL: Solubilized collagen with alkali. Triple helices of collagen molecules were substantially freed of the teropeptide at both terminals by alkali. In addition, several amino acid species were chemically modified.

gelatin: Gelatin. Irreversibly denatured collagen with heating. Characterized by substantial changes in physicochemical and biochemical properties.

no coat: Non-coated glass plate.

The data in Table 4 shows that even after 4 days of cell culture, SCEL maintained integrin expression at a substantially comparable level to SCE.

Thus, the above results demonstrate that the SCEL of the invention is comparable to the conventional types of water-soluble collagen in terms of suitability for use in cosmetics.

### Example 2

A dispersion of a collagen association product having high thermal denaturation temperature was prepared in accordance with the following recipe.

| | | |
|---|---|---|
| Purified collagen precipitate | 50 | g |
| (10 g of collagen on a dry weight basis) | | |
| Methylparaben | 1 | g |
| 1.2 N HCl | 9 | g |
| 1.0 N NaOH | 14 | g |
| Distilled water | 926 | g |
| Total | 1000 | g |

After dispersing 50 g of the purified collagen precipitate and 1 g of methylparaben in 926 g of distilled water, 9 g of 1.2 N HCl was added to adjust the dispersion to pH 2.8, followed by stirring to make a uniform solution. After passing the solution through a 0.65 μm membrane filter to remove the insoluble matter, 14 g of 1.0 N NaOH was added to adjust the solution to pH 7.0, thereby forming an association product. Subsequently, the association product was transferred into a 2-L cylindrical plastic container, where it was dispersed uniformly with a homogenizer to prepare a dispersion of the collagen association product, which started to denature thermally at 42.3° C., with a peak temperature of 48.6° C.

### Example 3

The dispersion of collagen association product prepared in Example 2 exhibited the properties of an emulsion (appearing white to milky white with low viscosity). The dispersion was immediately charged into a container to make a cosmetic in emulsion form.

### Example 4

A cosmetic (moisturizing jelly) containing a dispersion of collagen association product was prepared in accordance with the following recipe.

| | | |
|---|---|---|
| Purified collagen precipitate | 50 | g |
| (10 g of collagen on a dry weight basis) | | |
| Methylparaben | 1 | g |
| 1.2 N HCl | 9 | g |
| 1.0 N NaOH | 14 | g |
| Distilled water | 826 | g |
| Aqueous solution of sodium hyaluronate | 100 | g |
| Total | 1000 | g |

After dispersing 50 g of the purified collagen precipitate and 1 g of methylparaben in 826 g of distilled water, 9 g of 1.2 N HCl was added to adjust the dispersion to pH 2.8, followed by stirring to make a uniform solution. After passing the solution through a 0.65 μm membrane filter to remove the insoluble matter, 14 g of 1.0 N NaOH was added to adjust the solution to pH 7.0, thereby forming an association product. Subsequently, the association product was transferred into a 2-L cylindrical plastic container, where it was dispersed uniformly with a homogenizer to prepare a dispersion of the collagen association product. Thereafter, 100 g of the aqueous sodium hyaluronate solution was added to the dispersion, which was stirred with a mixer/stirrer until uniformity was obtained. The mixture was subjected to vacuum defoaming and charged into a container. The collagen in the thus prepared moisturizing jelly started to denature thermally at 42.3° C., with a peak temperature of 48.6° C.

Conventionally, collagenous substances have been difficult to be incorporated in cosmetics of high water content while holding their thermal denaturation temperature at high level. The present invention solves this problem and the quality control of cosmetic feeds and cosmetics at all stages including manufacture, storage and distribution can be performed easily as compared with the conventional collagen incorporating cosmetics.

What is claimed is:

1. A cosmetic feed containing a dispersion of a collagen association product that is obtained by adjusting an aqueous solution of collagen having a specified isoelectric point to a pH near said isoelectric point.

2. A cosmetic incorporating a cosmetic feed containing a dispersion of a collagen association product obtained by adjusting an aqueous solution of collagen having a specified isoelectric point to a pH near said isoelectric point.
